(19) United States
(12) Patent Application Publication
(10) Pub. No.: US 2010/0151001 A1
(43) Pub. Date: Jun. 17, 2010

(54) ETHYNYLATED HETERODINUCLEOSIDE PHOSPHATE ANALOGS, METHOD FOR THE PRODUCTION THEREOF, AND USE THEREOF

(75) Inventors: Herbert Schott, Tubingen (DE); Peter Ludwig, Reutlingen (DE)

Correspondence Address: BACON & THOMAS, PLLC, 625 SLATERS LANE, FOURTH FLOOR, ALEXANDRIA, VA, 22314-1176, US

(73) Assignee: Eberhard Karls Universitat Tubingen, Tubingen (DE)

(21) Appl. No.: 12/450,771

(22) PCT Filed: Apr. 10, 2008
(86) PCT No.: PCT/EP2008/054322

§ 371 Date: Feb. 18, 2010

(30) Foreign Application Priority Data
Apr. 13, 2007 (EP) 07007635.1

(51) Int. Cl. A61K 31/7072; C07H 19/10; A61K 9/127; A61K 9/14; A61P 35/00; A61P 31/00

(52) U.S. Cl. 424/450; 536/26.5; 514/51; 424/489; 977/773

(57) ABSTRACT

The invention relates to novel ethynylated heterodinucleoside phosphate analogs, the production thereof, substances containing at least one of said compounds, and the use thereof for the treatment of cancer and infectious diseases.

The present invention relates to novel active substances, production thereof, agents containing at least one of these compounds and use thereof for the treatment of cancers and infectious diseases.

### BACKGROUND OF THE INVENTION

Nucleoside analogs, possessing certain structural features, are proven medicinal products in the chemotherapy of cancers and virus-induced diseases (Advanced Drug Delivery Review (1996) 19, 287). Analogs of cytidine, for example 1β-D-arabinofuranosylcytosine (araC), or of uridine, for example 5-fluoro-2-deoxyuridine (5FdU), prevent DNA replication and are effective against malignant diseases of the hematopoietic cells and against solid tumors. For treatment of HIV infection, in particular dideoxynucleoside analogs are suitable, such as 3′-azido-2′,3′-dideoxythymidine (AZT), 2′,3′-dideoxycytidine (ddC), 2′,3′-dideoxyinosine (ddI), 3′-thia-2′,3′-dideoxycytidine (3TC) and 2′,3′-didehydro-2′,3′-dideoxythymidine (d4T). Nucleoside analogs with ethynyl residues, for example 3′-C-ethynylcytidine (ECyd), are multifunctional antitumor drugs with a broad spectrum of activity (Hattori, H. et al. J. Med. Chem. 1996, 39, 5005; Azuma, A. et al. Nucleosides, Nucleotides & Nucleic Acids, 2001, 20. 609)

The therapeutic action of nucleoside analogs requires the nucleoside analogs that are administered, which as a rule are inactive “prodrugs”, to be taken up by the cell and to be anabolized to the actual active substances, the 5′-triphosphate derivatives of the nucleoside analogs. The phosphorylated derivatives can impair DNA and/or RNA synthesis with lethal consequences for the cell, or can prevent virus replication.

Owing to the development of resistance that frequently occurs during chemotherapy with just one active substance (monotherapy), the medicinal product administered can lose its effectiveness in the course of the therapy. For a sustainable slowing of the progression of the disease and for effectively counteracting the development of resistance, various active substances are applied together (combination therapy). The application of a dosage form for HIV therapy (Schweiz. Med. Wochenschr. (1997), 127, 436) containing e.g. AZT and 3TC as a mixture, as in the case of “Combivir”, at best only makes the combination therapy more practicable for the patient. However, it is scarcely possible to achieve improved antiviral action with such mixtures, since there is neither an increase in the cell's uptake of the active substances applied as a mixture, nor is their anabolization to the corresponding triphosphate derivatives optimized.

However, the antiviral and/or cytostatic action of combination preparations can be optimized considerably if the various nucleoside analogs are coupled chemically in one dosage form. The type of coupling of the two monomeric active substances to form a new combination active substance is decisive for the therapeutic action of these combination preparations. Covalent coupling must ensure that in a desired metabolization, therapeutically effective metabolites can be released, which produce additive or even synergistic effects and if possible cancel mechanisms of resistance to the monomeric active substances.

Coupling of a lipophilic nucleoside analog with a hydrophilic nucleoside analog via a phosphodiester bridge results in amphiphilic dinucleoside phosphate analogs (EP-A-0642527). The coupling of different hydrophilic nucleoside analogs via a glycerol-lipid backbone leads to amphiphilic glyceryl nucleotides (DE-A-19855963 or WO-A-00/34298). The type of coupling selected in these combination preparations fulfills the stipulated requirement. Both combination preparations make a considerable contribution to improvement of the chemotherapy of neoplastic and viral diseases.

The broad spectrum of activity of nucleoside analogs that have an ethynyl residue has so far only been utilized on the basis of the glyceryl nucleotide analogs. A substantial disadvantage of combination preparations of this type is their high cost of synthesis, which is mainly due to the multistage synthesis of the glyceryl lipid backbone. Moreover, the resultant high molecular weight may hamper the distribution of these combination preparations and the associated targeting of the active substance in vivo, so that the therapeutic action, may be impaired.

Non-ethynylated 3′-5′- and 5′-5′-coupled duplex active substances with antitumor activity are known from Ludwig, P. S. et al., European Journal of Medical Chemistry 2005, 494-504.

To what extent an unnatural (i.e. 5′-5′) and in particular a natural (i.e. 3′-5′) phosphodiester bridge are suitable for the coupling of ethynylated nucleoside analogs with other therapeutically effective nucleoside-based compounds is largely unexplained, as the influence of an ethynyl residue on the metabolization of a dimer has not been elucidated. It cannot be ruled out that an ethynylated nucleoside at the 3′-end of the dimer prevents the hydrolytic removal of the ethynylated building block, since the 3′-end is masked for exonucleases by the ethynyl residue, so that the desired metabolization of the duplex active substance to the two monomeric active substances would not be able to occur.

Immunoliposomes, which are directed against the tumor marker TEM1 and for this purpose are functionalized with a special antibody fragment (ScFv-CM6), are known from Marty, C. et al., Cancer Letters 2006, 235, 298-308. In addition, loading with the cytotoxic active substance N⁴-octadecyl-1-β-D-arabinofuranosylcytosine-(5′-5′)-3′-C-ethynylcytidine has been proposed. However, such a preparation is designed to transport the active substance to the tumor directly, i.e. without metabolic cleavage. Suitability as a medicinal product for classical, i.e. oral or intraperitoneal administration, for example, cannot yet be concluded from this.

### SUMMARY OF THE INVENTION

The problem to be solved by this invention is to provide novel, easily accessible combination preparations, with which cancers and/or viral diseases can be treated in a novel way.

This problem is solved, surprisingly, with novel dinucleoside phosphate analogs, which upon metabolization simultaneously release several, variously active nucleoside analogs, for example with different mechanisms of action, of which always at least one nucleoside analog bears the therapeutically highly effective ethynyl residue. With these so-called duplex active substances, not only the general advantages of a combination therapy, but for the first time also the multifunctional efficacy of ethynylated nucleoside analogs can be utilized in combination preparations. The necessary, determining phosphorylation step for activation of the ethynylated nucleoside analogs by the body's own kinases, such as uridine/cytidine-kinase, can be omitted with the duplex active substances, because during their metabolization the ethynylated nucleoside, for example ECyd, can already be formed in the phosphorylated form. Furthermore, compared with the ethynylated glyceryl nucleotide analogs, the cost of synthesis is considerably less for the dinucleoside phosphate analogs.

Surprisingly, moreover, a class of ethynylated dinucleoside phosphate analogs preferred according to the invention, which are coupled via a natural 3′-5′-phosphodiester bridge or analogous end-ring couplings, and in particular those bearing the ethynylated monomer via its 5′-position at the 3′-end of the nonethynylated second monomer, display significantly greater antitumor activity than the corresponding isomers that have an unnatural 5′-5′-coupling. The clear superiority of the 3′-5′-coupling can be demonstrated on the basis of the concentrations of active substances determined for total inhibition of growth of the tumor cells (see example 3, TGI values). The TGI values for the 3′-5′-coupled isomer are often up to 1000-times lower compared with the values for the 5′-5′-coupled isomer. The manner of coupling is, surprisingly, also decisive for a further improvement in efficacy, as presumably active substances with very different activity are formed during enzymatic metabolization. By choosing the direction of the phosphodiester bridge, the antitumor activity of a combination preparation can, surprisingly, be modulated so that the spectrum of action of the ethynylated duplex active substances can be further improved. Furthermore, the natural 3′-5′-coupling, compared with the unnatural 5′-5′-coupling, is considerably more easily accessible by synthesis and is preferred when the molecular structure of the monomers to be coupled permits natural 3′-5′-phosphodiester binding.

### DETAILED DESCRIPTION OF THE INVENTION

### a) General Concepts

Unless stated otherwise, according to the invention both individual isomers of active substances according to the invention and any mixtures of stereoisomeric forms thereof are also included. In particular all stereoisomeric forms of compounds according to the invention in pure form and any mixtures of said stereoisomeric forms are also included.

Furthermore, dinucleoside phosphate analogs according to the invention can comprise any combinations of D- and L-isomers of their nucleoside building blocks.

Furthermore, according to the invention all possible diastereomeric or anomeric forms of compounds according to the invention, in particular alpha- and beta-anomers, are also included.

Nucleoside residues comprise, according to the invention, natural nucleosides, such as adenosine, guanosine, cytidine, thymidine, uridine, inosine, the corresponding mono- and dideoxy forms and structurally analogous compounds, obtainable by changing the glycosidic residue and/or the basic residue, as explained in more detail below.

The terminal coupling of a nucleoside usually takes place via an HOCH₂ or HSCH₂ group, whereas cyclic coupling usually takes place via a —CH(OH)- or —CH(SH)-group of the glycosidic residue.

Compounds according to the invention comprise, depending on the nature of the optionally used substituents, compounds of an amphiphilic, lipophilic or hydrophilic character.

According to the invention, treatment of a disease comprises both prophylaxis and, in particular, therapy.

### b1) Heterodinucleoside Phosphate Analogs According to the Invention

The invention relates in particular to ethynylated heterodinucleoside phosphate analogs of formula I

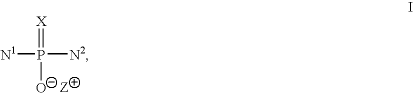

in which
X stands for O or S;
Z stands for H or the corresponding salt of acid addition of this compound;
N¹ and N² are different and in each case stand for a nucleoside group, with each of the nucleoside groups, which in each case have a glycosidic residue or cyclic residue derived therefrom and a basic residue coupled covalently to it, being joined covalently via their glycosidic residue to the central P-atom, in particular coupled or bridged with oxygen or sulfur; and with at least one of the nucleoside groups having an ethynylated glycosidic residue. In particular the coupling is a coupling that can be cleaved enzymatically, in particular in vitro or in vivo, in the human body. Furthermore, the coupling of the glycosidic residues can be terminal-terminal (end-to-end coupling, for example 5′-5′) or terminal-cyclic (end-to-ring coupling, for example 3′-5′). In particular, bridging is terminal-cyclic (end-to-ring coupling, for example 3′-5′). “Cyclic” coupling takes place by bridging of a ring-carbon atom of the glycosidic ring or ring of the nucleoside derived therefrom with the P-atom, for example via the 3′-carbon atom of a pentose. “Terminal” coupling takes place by bridging a terminal, nonring-carbon atom of the glycosidic ring or ring of the nucleoside derived therefrom with the P-atom, for example via the terminal 5′-carbon atom of a pentose. The terms “end-to-ring” and “ring-to-end” are to be understood as synonymous terms, and are not bound to a particular order. This applies correspondingly to the terms “3′-5′” and “5′-3′”. The positioning of the ethynyl substituent is also not fixed as the end-coupled or ring-coupled nucleoside.

The invention relates in particular to compounds of formula I, each of the identical or different, optionally ethynylated glycosidic residues of N¹ and N², which is in particular in the form of a pyranoside or furanoside residue, being derived from a pentose, hexose or heptose, with one or more ring-bound H atoms or hydroxyl groups optionally being eliminated or substituted with halogen, hydroxyl, cyano, 2-fluoromethylene, trifluoromethyl or azido; optionally a heteroatom, selected from S, N and O instead of a ring-carbon atom, can be contained in the glycosidic residue; and the glycosidic residue can optionally contain one or two nonadjacent C═C double bonds.

The invention also relates in particular to compounds of formula I, each of the identical or different basic residues being the residue of a mono- or binuclear heterocyclic base, which is constructed from one or two four- to seven-membered rings, the basic residue containing at least one basic ring-N atom and optionally at least one basic amino group and optionally at least one further ring-heteroatom, selected from S and O; and with the basic residue optionally substituted one or more times, for example 1, 2, 3, 4, 5 or 6 times, with hydroxyl, amino, halogen, alkyl, alkenyl, polyoxyalkenyl, aryl, acyl, alkyloxy, alkenyloxy, polyoxyalkenyloxy, acyloxy, aryloxy, alkylthio, alkenylthio, acylthio or arylthio; the amino, alkyl, alkenyl and acyl residues optionally being substituted with 1, 2 or 3 aryl residues, polyoxyalkylene residues or halogen atoms.

### b2) Definitions Of General Residues

The glycosidic residue of the nucleoside or nucleoside derivative is derived from a hexose or heptose, though preferably from a pentose, for example deoxyribose, dideoxyribose or ribose. In the glycosidic residue, optionally individual or several protons or hydroxyl groups can be substituted or eliminated. Suitable substituents are selected from hydrogen, halogen, such as F, Cl, Br and J, hydroxyl, ethynyl, trifluoromethyl, cyano, 2-fluoromethylene, and azido. Optionally, a heteroatom, selected from S, N and O, can be contained instead of a carbon atom and optionally the sugar residue can contain one or two nonadjacent C═C double bonds.

The basic moiety of the nucleoside or nucleoside derivative is the residue of a mono- or binuclear heterocyclic base, composed of one or two four- to seven-membered rings, which together contain at least one ring-heteroatom, for example one to six heteroatoms, selected from N, S and O, in particular N and O. Examples of such bases are the purine and pyrimidine bases adenine, guanine, cytosine, uracil and thymine. Further examples of usable bases are pyrrole, pyrazole, imidazole, aminopyrazole, 1,2,3-triazole, 1,2,4-triazole, tetrazole, pentazole, pyridone, piperidine, pyridine, indole, isoindole, pyridazine, indoxyl, isatin, pyrazine, piperazine, gramine, tryptophan, kynurenic acid, tryptamine, 3-indolylacetic acid, carbazole, indazole, 1,3,5-triazine, 1,2,4-triazine, 1,2,3-triazine and tetrazine. Preferred bases are adenine, guanine, cytosine, uracil and thymine; and 1,2,3-triazole, 1,2,4-triazole and tetrazole. The stated bases can optionally be substituted one or more times, for example one to four times, in particular once or twice, with the aforementioned residues hydroxyl, amino, halogen, alkyl, alkenyl, polyoxyalkenyl, aryl, acyl, alkyloxy, alkenyloxy, polyoxyalkenyloxy, acyloxy, aryloxy, alkylthio, alkenylthio, acylthio or arylthio, with the alkyl, alkenyl and acyl optionally substituted with 1 to 3 aryl residues or halogen atoms. The substitution can take place on a ring-heteroatom or preferably on a ring-carbon atom or a side group, for example an amino side group of the base.

Compounds according to the invention can in addition be substituted selectively with a lipophilic residue on one or both, preferably one of the nucleoside groups. The lipophilic residue should be a linear or branched hydrocarbon residue, in particular alkyl, alkenyl, acyl, alkyloxy, acyloxy, aryloxy, alkenyloxy, alkylthio, alkenylthio, acylthio or arylthio residue, as defined below, and should preferably comprise more than 6, for example 7 to 30 or 10 to 24 carbon atoms.

The following may be mentioned as examples of suitable aryl residues: phenyl, naphthyl, and benzyl.

As examples of suitable alkyl residues, we may mention linear or branched residues with 1 to 24 carbon atoms, such as methyl, ethyl, i- or n-propyl, n-, i-, sec.- or tert.-butyl, n- or i-pentyl; in addition n-hexyl, n-heptyl, n-octyl, n-nonyl, n-decyl, n-undecyl, n-tridecyl, n-tetradecyl, n-pentadecyl and n-hexadecyl, octadecyl, docosanyl, and the singly or multiply branched analogs thereof.

Examples of suitable alkenyl residues are the singly or multiply, preferably singly or doubly, unsaturated analogs of the aforementioned alkyl residues with 2 to 24 carbon atoms, the double bond being located in any position of the carbon chain.

Examples of suitable polyoxyalkenyl residues are derived from C₂-C₄ alkylene oxides, which can comprise 2 to 12 recurring alkylene oxide units.

Examples of suitable acyl residues are derived from linear or branched, optionally singly or multiply unsaturated, optionally substituted C₁-C₂₄ monocarboxylic acids. For example, usable acyl residues are derived from the following carboxylic acids: saturated acids, such as formic, acetic, propionic and n- and i-butyric acid, n- and i-valeric acid, hexanoic acid, oenanthic acid, octanoic acid, pelargonic acid, capric acid, undecanoic acid, lauric acid, tridecanoic acid, myristic acid, pentadecanoic acid, palmitic acid, margaric acid, stearic acid, nonadecanoic acid, arachidic acid, behenic acid, lignoceric acid, cerotinic acid and melissic acid; singly unsaturated acids, such as acrylic acid, crotonic acid, palmitoleic acid, oleic acid and erucic acid; and doubly unsaturated acids, such as sorbic acid and linoleic acid. If the fatty acids contain double bonds, these can be both in the cis and in the trans form.

Examples of suitable alkyloxy, acyloxy, aryloxy, alkenyloxy and polyoxyalkylene-oxy residues are the oxygen-terminated analogs of the aforementioned alkyl, acyl, aryl, alkenyl and polyoxyalkylene residues.

Examples of suitable alkylthio, alkenylthio, acylthio or arylthio residues are the corresponding sulfur-terminated analogs of the above alkyloxy, alkenyloxy, acyloxy and aryloxy residues.

### b3) Preferred Compounds

The invention also relates in particular to compounds of formula I, with N¹ and N² being P-coupled via identical or different positions of the glycosidic groups.

The invention also relates in particular to compounds of formula I, with each of the identical or different glycosidic residues being a furanoside residue or five-membered residue derived therefrom; in particular, N¹ and N² are linked together 3′-5′ or 5′-5′ via the P-atom.

In particular, compounds are preferred in which the terminally coupled, in particular the 5′-coupled furanoside residue of one nucleoside bears the ethynylation, in particular in the 3′- or 2′-position of the furanoside ring. The ring-coupled, in particular 3′-coupled, furanoside residue of the other nucleoside is, in contrast, not ethynylated.

The invention also relates in particular to compounds of formula I, in which X and Z have the meanings given above and the groups N¹ and N² are different from one another and stand for a D- or L-configured nucleoside derivative of formula II, III, and IV

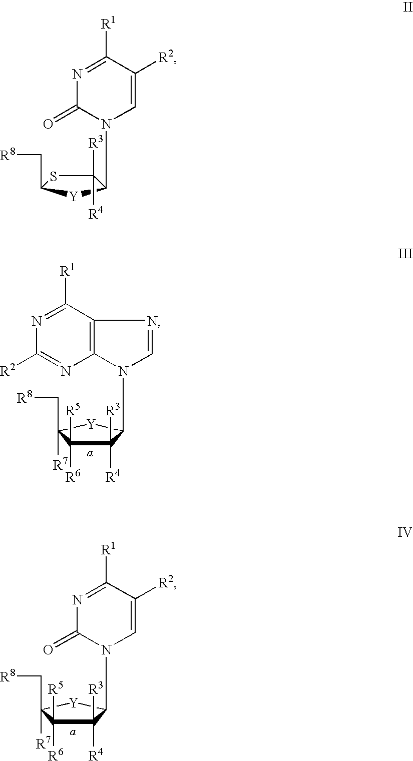

in which
Y stands for O or S;
R¹ represents a hydroxyl, alkoxy, amino, acylated, alkylated or polyoxyethylene-substituted amino group, whose acyl or alkyl residue is linear or branched, has 1 to 24 carbon atoms and up to 2 double bonds and can be substituted with 1, 2 or 3 aromatic residues or a heterocycle,
R² stands for H, halogen, an amino, hydroxyl or trifluoromethyl group, a bromovinyl, a linear or branched C₁-C₂₄ alkyl residue;
R³ to R⁸ are identical or different, and stand for H, halogen, hydroxyl, ethynyl, cyano, fluoromethylene, trifluoromethyl or azido, with two of the residues R³ to R⁶ being omitted if the C—C bond in position “a” stands for a double bond;
N¹ and N² being selected in such a way that always one of the residues R³ to R⁸ of N¹ and N², independently of one another, stands for —O— or —S—, by which N¹ and N² are coupled to the central P-atom of formula I; and at least one of the remaining residues R³ to R⁸ in N¹ or N² denotes ethynyl, so that either N¹ or N² has at least one ethynyl residue.

The invention also relates in particular to compounds of formula I, in which X, Y, Z and “a” have the meanings given above and

a) in the groups N¹ and N² independently of one another
R¹ stands for an alkylated or acylated amino group according to the above definition, in particular stands for an alkylated or acylated amino group in which its alkyl residue is a hexadecyl residue and the acyl residue is a palmitoyl, oleoyl or behenoyl residue;
R² stands for H, halogen, methyl, ethyl or trifluoromethyl;
R³, R⁴ and R⁷ stand for azido, H, fluoro, fluoromethylene, cyano, trifluoromethyl or hydroxyl;
and simultaneously
b) in one of the groups N¹ and N²
R⁵ stands for ethynyl and in the other groups. R⁵, if present, stands for azido, H, fluoro or hydroxyl;
and simultaneously
c) in each of the groups N¹ and N² independently of one another
one of the residues R⁶ and R⁸ stands for —O— or —S— and the other of the two residues R⁶ and R⁸ stands for azido, H, fluoro or hydroxyl.

In particular, the invention relates to compounds of formula I in which X, Y, Z and “a” have the meanings given above and

a) in the groups N¹ and N² independently of one another
R¹ stands for an alkylated or acylated amino group according to the above definition;
R² stands for H, halogen, methyl, ethyl or trifluoromethyl;
R³, R⁴ and R⁷ stand for azido, H, fluoro, fluoromethylene, cyano, trifluoromethyl or hydroxyl;
and simultaneously
b) one of the groups N¹ and N² is ethynylated, and in said group R⁵ stands for ethynyl and in the nonethynylated group R⁵, if present, stands for azido, H, fluoro or hydroxyl;
and simultaneously
c) in the ethynylated group of N¹ and N²
the residue R⁸ stands for —O— or —S— and the residue R⁶ stands for azido, H, fluoro or hydroxyl; and in the nonethynylated group R⁶ stands for —O— or —S— and the residue R⁸ stands for azido, H, fluoro or hydroxyl.

Other groups of preferred compounds are:
(1) Compounds
   in which Z and “a” have the meanings given above
   X stands for O;
   N¹ and N² are different and stand for a nucleoside derivative of formula IV, in which Y stands for O;
   R¹ stands for an amino, C₁₂-C₂₂ alkylamino, C₁₂-C₂₂ acyl-amino group or a hydroxyl group;
   R² stands for H, fluoro or trifluoromethyl; and
   R³ to R⁸ have the meanings given above.
(2) Compounds in which N¹ stands for a, preferably not ethynylated nucleoside residue of formula IV, in which the residues R¹ to R⁸ have the meanings given above and N² stands for a nucleoside residue of formula IV, which is ethynylated, in which
   R¹ stands for amino
   R², R³, R⁷ for H;
   R⁴, R⁶ for hydroxyl;
   R⁵ for ethynyl; and
   R⁸ for an oxygen atom, with which N² is bridged with the P-atom.
   In particular they are compounds in which N¹ stands for an optionally ethynylated nucleoside residue of formula IV, in which the residues R¹ to R⁵, R⁷ and R⁸ have the meanings given above and R⁶ stands for an oxygen atom, with which N¹ is bridged with P, and N² stands for a nucleoside residue of formula IV, which is ethynylated, in which
   R¹ stands for amino
   R², R³, R⁷ for H;
   R⁴, R⁶ for hydroxyl;
   R⁵ for ethynyl; and
   R⁸ stands for an oxygen atom, with which N² is bridged with P.
      Compounds in which N² stands for a nucleoside residue of formula IV, which is ethynylated, and N¹ stands for a nucleoside residue of formula IV, which is not ethynylated, in which
   R¹ stands for hexadecyl, palmitoyl, oleoylamino or hydroxyl;
   R² stands for H or fluoro;
   R³, R⁴ are identical or different and stand for H, hydroxyl, fluoro;
   R⁵, R⁷ stand for H;
   R⁶ and R⁸ independently of one another stand for hydroxyl, azido or H, with the proviso that one of the residues stands for —O—.
   In particular they are compounds in which N² stands for a nucleoside residue of formula IV, which is ethynylated, and N¹ stands for a nucleoside residue of formula IV, which is not ethynylated, in which
   R¹ stands for hexadecyl, palmitoyl, oleoylamino or hydroxyl;
   R² stands for H or fluoro;
   R³, R⁴ are identical or different and stand for H, hydroxyl, fluoro;
   R⁵, R⁷ stand for H;
   R⁶ stands for —O— and R⁸ stands for hydroxyl, azido or H.
(3) Compounds, in which N² stands for a nucleoside residue of formula IV, which is ethynylated, and N¹ stands for a nucleoside residue of formula IV, which is not ethynylated, in which
   R¹ stands for hydroxyl;
   R² stands for fluoro;
   R³, R⁴, R⁵ and R⁷ stand for H,
   R⁶ stands for —O—, and
   R⁸ stands for hydroxyl.

Preferred classes of heterodinucleoside phosphate analogs comprise in particular, as ethynylated component, a nucleoside residue of a 2′-, 3′ or 4′-C-ethynyl nucleoside, such as in particular of 2′-, 3′ or 4′-C-ethynylcytidine or 2′-, 3′ or 4′-C-ethynyluridine, in particular 2-, or 3′-C-ethynylcytidine.

Preferred individual compounds are selected from:
5-fluoro-2′-deoxyuridylyl-(3′-5′)-3′-C-ethynylcytidine
arabinocytidylyl-(5′-5′)-3′-C-ethynylcytidine
N⁴-hexadecylarabinocytidylyl-(5′-5′)-3′-C-ethynylcytidine
(E)-2′-deoxy-(2-fluoromethylene)cytidylyl-(3′-5′)-3′-C-ethynylcytidine
β-L-dioxolanecytidylyl-(5′-5′)-3′-C-ethynylcytidine
2′-C-cyano-2′-deoxyarabinocytidylyl-(3′-5′)-3′-C-ethynylcytidine
2-chloro-(2′-deoxy)-fluoroarabinoadenylyl-(3′-5′)-3′-C-ethynylcytidine
2′-deoxy-2′,2′-difluorocytidylyl-(3′-5′)-3′-C-ethynylcytidine

### c) Production of Heterodinucleoside Phosphate Analogs According to the Invention

In addition, the invention relates to methods of production of ethynylated heterodinucleoside phosphate analogs according to the invention, in which two nucleosides of general formulas Va and Vb

L¹-N¹ (Va)

L²-N² (Vb)

in which
N¹ and N² are as defined above and optionally have one or more protecting groups, in particular with at least one of the groups N¹ and N² bearing an ethynyl or protected ethynyl group on the glycosidic residue; and
L¹ and L² represent groups that are bound on the glycosidic residue of N¹ and N² and are reactive with one another, where one of the groups L² and L² stands for a hydroxy or mercapto group and the other stands for a hydrogenphosphonate or thiohydrogenphosphonate group; and where in particular one of the groups L¹ and L² is bound cyclically and the other is bound terminally;
are condensed in the presence of an acid chloride and the condensation product is then oxidized, in particular in order to oxidize the phosphonate bridge that formed in the condensation to the phosphate bridge, and any optionally present protecting groups are removed.

“Cyclic” binding takes place by binding to a ring-carbon atom of the glycosidic or ring of the nucleoside derived therefrom, for example with the 3′-carbon atom of a pentose. “Terminal” binding takes place by binding to a terminal, nonring-carbon atom of the glycosidic or ring of the nucleoside derived therefrom, for example with the terminal 5′-carbon atom of a pentose.

It relates in particular to a method of production, characterized in that in each case two nucleosides of the formulas Va and Vb are reacted, the nucleosides Va and Vb corresponding to a compound of the above general formula II, III or IV, in which X and “a” have the meanings given above,

L¹ and L² are contained instead of one of the residues R³ to R⁸, in particular R⁶ or R⁸, the residues R¹ to R⁸ otherwise have the meaning given above;
the residues R¹ and R³ to R⁸ can additionally also stand for an acylated hydroxyl group, whose acyl residue is linear or branched, has 1-24 carbon atoms and 1 or 2 double bonds and can be substituted with an aromatic residue, or can stand for tert-butyldimethylsilyloxy protecting group,
R⁸ additionally can also stand for a 4-mono-, or 4,4′-dimethoxytriphenylmethyloxy protecting group;
and at least one of the residues R³ to R⁸, in particular residue R⁵ can also stand for trimethylsilylethynyl.

Furthermore it is preferable for the optionally present 4-mono- or 4,4′-dimethoxytriphenylmethyloxy protecting groups to be exchanged for hydroxyl, and for acyl and silyl residues optionally to be cleaved hydrolytically.

Unless stated otherwise, production of compounds according to the invention is carried out using methods or synthesis techniques that are known per se and are familiar to a person skilled in the art in the area of the synthesis of nucleoside analogs.

The condensation is especially successful in solution in the presence of acid anhydrides or acid halides, such as in particular pivalic acid chloride, at −80° C. to +100° C., for example at about 0-20° C. A suitable solvent is e.g. pyridine.

The oxidation is especially successful in solution at −80° C. to +100° C., for example at about 0-20° C., oxidizing a) the P—H bond to a P═O bond with iodine in aqueous organic solvents or b) the P—H bond to a P═S bond with S₈ in triethylamine/CS₂. A suitable solvent is e.g. THF.

After oxidation and chromatographic processing, separation of the protecting groups is carried out in a way that is known per se. For example, the 4-mono- or 4,4′-dimethoxytriphenylmethyl group is exchanged for hydroxyl, and/or trimethylsilyl for hydrogen, and/or acyl residues are if necessary converted hydrolytically to mercapto, hydroxyl and/or amino groups.

The starting materials required for the reactions are substances that are known per se or can be produced by analogy with known methods (Antivir. Chem & Chemother. (1998) 9, 33; Makromol Chem. (1986) 187, 809; Tetrahedron Lett. (1986) 27, 2661; Synthesis (2002) 16, 2387; Eurp. J Med. Chem. (2005) 40, 494); to which reference is hereby expressly made.

In particular the ethynylated nucleoside building blocks used according to the invention are also known per se or can easily be produced (also see, for example, Bioorg. Med. Chem. (2005) 13, 2597-2621; Cancer Sci (2005), 96, 5, 295-302; J. Med. Chem. (1996) 39, 5005-5011; Radiation Research (2004) 162, 635-645); to which reference is hereby expressly made.

For example, the condensation of two nucleosides derivatives to the duplex active substance according to the invention can be carried out as follows:

A first protected nucleoside derivative bearing a hydrogenphosphonate group (for example 5′-O-(4-monomethoxytrityl)-5-fluoro-2′-deoxyuridine-3′-hydrogenphosphonate) is dissolved, together with a second nucleoside derivative bearing a protected ethynyl group and optionally other protecting groups (for example N⁴-benzoyl-2′-O-(tert.-butyldimethylsilyl)-3′-C-(trimethylsilylethynyl)cytidine) in an anhydrous polar solvent, for example pyridine. A suitable condensation aid, such as an acid chloride, for example pivaloyl chloride, is added to the solution cooled to approx. 0 to 15° C., with exclusion of moisture, after thorough mixing and reaction at room temperature it is cooled again and water is added. Then a solution of iodine, e.g. in tetrahydrofuran, is added to the reaction mixture and it is reacted again at room temperature. Excess iodine is reduced by adding NaHSO₃, before the reaction mixture is concentrated, which is then taken up in an organic solvent, such as a chloroform/methanol mixture and is extracted with water. The organic phase is concentrated and chromatographed, e.g. by diluting with chloroform and then fractionating in a silica gel column with a chloroform/methanol gradient, with increasing proportion of methanol.

For complete replacement of optionally present 5′-hydroxy protecting groups (e.g. monomethoxytrityl group) with hydrogen, the product obtained is stirred in methanol/acetic acid at room temperature and then concentrated again. Ether is added to the residue so that it is converted to a precipitate, which is centrifuged and dried and chromatographed again as above. The product is dried and, for replacement of optionally present silyl groups with hydrogen, it is dissolved in a dry organic solvent, such as tetrahydrofuran, tetrabutylammonium fluoride trihydrate e.g. in tetrahydrofuran is added, it is stirred at room temperature and then concentrated.

For replacement of optionally present amino protecting groups (such as benzoyl groups) with hydrogen, concentrated ammonia solution is added to the product obtained and it is stirred at room temperature. The mixture is then concentrated and the product is isolated, dissolved in water and fractionated by reverse phase chromatography (e.g. RP-18 column) e.g. with a water/methanol gradient, with increasing proportion of methanol. The fractions containing the product are combined, adjusted with a cation exchanger (H⁺ form) e.g. to pH approx. 5.8 and separated from the exchanger again. The filtrate is neutralized with ammonia, concentrated and then lyophilized, obtaining the desired duplex active substance.

### d) Pharmaceutical Formulations and Uses According to the Invention

Coupling of the ethynylated nucleoside, such as the therapeutically highly effective ethynylcytidine, to a second, also effective nucleoside analog, results in a so-called duplex active substance, which displays additive and/or synergistic mechanisms of action. This effect is especially strongly pronounced when both monomers attack different targets. As a result, the applied therapeutic amount of the high-potency duplex active substances in comparison with that for the respective monomers can be dosed so that the desired therapeutic action is optimized, and simultaneously the undesirable toxic side effects are reduced decisively.

The different sequence, a natural 3′-5′ or unnatural 5′-5′ phosphodiester bridge, in which both nucleoside analogs can be coupled to the heterodinucleoside phosphate analogs, leads in enzymatic metabolization in vivo to very differently, predeterminable derivatized metabolites. As a result, the therapeutic spectrum can be almost programmed and expanded decisively. One consequence is that duplex active substances are effective for resistances against which the respective monomeric nucleoside analogs prove to be ineffective.

The conversion of ethynylated nucleosides, such as ethynylcytidine, to duplex active substances also gives rise to a greatly altered pharmacokinetic behavior, which in turn contributes to optimization of therapy. Owing to the considerable variability of derivatization, ethynylated duplex active substances can be prepared with very different solubility properties, depending on the type of substituents introduced. This opens up numerous possibilities for pharmaceutical formulation, which cannot be used for the monomeric ethynylcytidine, on account of its hydrophilicity.

Another advantage of the duplex active substances according to the invention is that, together with one or more other active substances, they can be incorporated in varying amounts in liposomes or nanoparticles, leading to synergistic effects.

The invention also relates to pharmaceutical agents, containing at least one compound according to the above definition in a pharmaceutically compatible vehicle or diluent, such as in particular contained in liposomes or nanoparticles.

Furthermore, agents according to the invention can additionally contain at least one other pharmacological active substance, which is suitable for the treatment of infectious diseases and/or cancers.

We may mention, as nonlimiting examples of other active substances for tumor treatment:

(A) Antineoplastic agents, such as
(1) phytocytostatics, e.g. mistletoe preparations,
(2) chemically defined cytostatics, such as
a) alkaloids and podophyllotoxins, for example vinblastin, vincristin and other vinca alkaloids and analogs; podophyllotoxin derivatives, such as etoposide;
b) alkylating agents, such as nitrosoureas and nitrogen mustard analogs, for example cyclophosphamide and estramustine;
c) cytotoxic antibiotics, such as anthracyclines and related substances, for example daunorubicin, doxorubicin; bleomycin and mitomycin;
d) antimetabolites, such as folic acid analogs, for example methotrexate, purine analogs, pyrimidine analogs, for example cytarabin and fluorouracil;
   (3) platinum compounds, such as carboplatin, cisplatin;
   (4) enzymes and monoclonal antibodies;
   (5) endocrine-active antineoplastics, such as
a) hormones and related substances, for example estrogens, gestagens, for example medroxyprogesterone acetate; hypothalamus hormones, such as gonadorelin analogs, for example buserelin;
b) hormone antagonists, such as the antiestrogen tamoxifen and other antiestrogens; or the antiandrogen flutamide and other antiandrogens;
c) enzyme inhibitors
   (B) Protective agents/antidotes for antineoplastic therapy, e.g. folinic acid.

As nonlimiting examples of other active substances for the treatment of infectious diseases, such as in particular AIDS, we may mention: azidothymidine, dideoxycytidine, sanilvudine, stavudine (1-(2,3-dideoxy-beta-D-glycero-pent-2-enofuranosyl)-5-methyl-2,4(1H,3H)-pyrimidinedione), dideoxyinosine, recombinant (human) interleukin-2, saquinavir mesylate, interferon alpha, nevirapine, abacavir sulfate, CD4-immunoadhesin, lamivudine, kynostatin-272, emtricitabine, delavirdine mesylate, HIV-1-immunogen, indinavir sulfate, azidothymidine phosphonate, calanolide A, amprenavir, efavirenz, ritonavir, nelfinavir mesylate, gadolinium texaphyrin, enfuvirtide, buffy coat interleukin, semapimod hydrochloride, elvucitabine, canovirin N, tipranavir, azodicarbonamide, tenofovir disoproxil fumarate, atazanavir sulfate, lamivudine/zidovudine, sampidine, dapivirine, etravirine, lopinavir/ritonavir, adargileukin-alpha, glyminox, ancriviroc, O-(2-hydroxypropyl)-beta-cyclodextrin, darunavir, maraviroc, abacavir sulfate/lamivudine, sulfonated hesperidin, rilpivirin, tenofovir,

In particular the invention also relates to the use of at least one compound according to the above definition for the production of a pharmaceutical agent for the prevention and/or therapy of infectious diseases and/or cancers.

The compounds according to the invention are generally used in the form of pharmaceutical agents for the treatment of an individual, preferably a mammal, in particular a human being. Thus, the compounds are usually administered in the form of pharmaceutical compositions, which comprise a pharmaceutically compatible excipient with at least one ethynylated nucleoside phosphate analog according to the invention, optionally also a mixture of several compounds according to the invention, and optionally other active substances that can be used for the respective desired therapeutic effect. Said compositions can for example be administered by the oral, rectal, transdermal, subcutaneous, intravenous, intramuscular or intranasal route.

Examples of suitable pharmaceutical formulations are solid pharmaceutical forms, such as powders, granules, tablets, pastilles, sachets, cachets, dragees, capsules such as hard and soft gelatin capsules, suppositories or vaginal pharmaceutical forms; semi-solid pharmaceutical forms, such as ointments, creams, hydrogels, pastes or plasters, and liquid pharmaceutical forms, such as solutions, emulsions, in particular oil-in-water emulsions, suspensions, for example lotions, preparations for injection and infusion, eye and ear drops. Implanted delivery devices can also be used for administration of the compounds according to the invention. Liposomes, microspheres or polymer matrixes can also find application.

For production of the pharmaceutical agents, compounds according to the invention are usually mixed or diluted with an excipient. Excipients can be solid, semi-solid or liquid materials, which serve as vehicle, carrier or medium for the active substance.

Suitable excipients include for example lactose, dextrose, sucrose, sorbitol, mannitol, starches, acacia gum, calcium phosphate, alginates, tragacanth, gelatin, calcium silicate, microcrystalline cellulose, polyvinylpyrrolidone, cellulose, water, syrup and methylcellulose. The formulations can also comprise pharmaceutically acceptable vehicles or usual excipients, such as glidants, for example tallow, magnesium stearate and mineral oil; wetting agents; emulsifying and suspending agents; preservatives, such as methyl- and propylhydroxybenzoates; antioxidants; antiirritants; chelating agents; sugar-coating aids; emulsion stabilizers; film-forming agents; gelling agents; odor-masking agents; flavor correctants; resins; hydrocolloids; solvents; solubilizers; neutralizing agents; permeation accelerators; pigments; quaternary ammonium compounds; refatting and overfatting agents; bases for ointments, creams or oils; silicone derivatives; spreading aids; stabilizers; sterilizing agents; bases for suppositories; tableting excipients, such as binders, fillers, glidants, disintegrants or coatings; propellants; drying agents; opacifiers; thickeners; waxes; plasticizers; white oils. An embodiment in this respect is based on expert knowledge, as described for example in Fiedler, H. P., Lexikon der Hilfsstoffe fur Pharmazie, Kosmetik and angrenzende Gebiete (Encyclopedia of excipients for pharmacy, cosmetics and related areas), 4th edition, Aulendorf: ECV-Editio-Kantor-Verlag, 1996.

Preferred usual vehicles are for example mannitol, glucose, dextrose, albumins or the like; preferred diluents are essentially physiological saline or a 5% glucose solution. Furthermore, it is usual to buffer such solutions with suitable reagents, for example phosphates.

For better application of the compounds according to the invention, compositions can be provided that contain the compounds according to the invention in combination with an organic vehicle. Furthermore, any other excipients that are usually employed for the preparation of pharmaceutical agents can be added, provided proper use of said composition of organic vehicle and the compounds according to the invention is not impaired.

A preferred embodiment of such compositions envisions the association of the compounds according to the invention in the form of uni- to oligolamellar liposomes with a diameter of max. 0.4 μm. All methods of liposome preparation that are known per se can be used for forming the liposomes, for example ultrasound, gel chromatography, detergent analysis, high-pressure filtration. The lipophilic residues introduced in each case have a decisive influence on the size and stability of the liposomes that form from the respective glyceryl nucleotides together with other lipid components (cf. Liposomes: From Physical Structure to Therapeutic Applications in: Research monographs in cell and tissue physiology Vol. 7, G. G. Knight Ed., Elsevier (1981).

Another preferred possibility for combining the compounds according to the invention with an organic vehicle is inclusion of the compounds in biologically compatible nanoparticles. The nanoparticles are organic-chemical polymers, to which the compounds according to the invention are added during polymerization, so that they are enclosed with a certain efficiency in the nanoparticle (cf. Bender et al., Antimicrobial agents and Chemotherapy (1996), 40 (6) 1467-1471).

In a preferred embodiment the composition according to the invention or the active substance according to the invention can comprise further components or be combined therewith, which promote specific enrichment in the region of the cells and/or organs to be treated. For example, the composition of the liposomes can be selected so that the liposomes are additionally provided with molecules, for example antibodies, charged lipids, or lipids modified with hydrophilic head groups, so that there is preferential enrichment of the composition in the cells and/or organs to be treated or in their vicinity. Such a composition, with molecules specifically directed against tumor cells, virus-infected cells and/or organs, increases the therapeutic action of the medicinal product and at the same time reduces the toxicity for uninfected tissues.

Such compositions can be processed into a pharmaceutical agent, which in addition to the compounds according to the invention and optionally the organic vehicle, also contains usual vehicles and/or diluents and/or excipients. Usual vehicles are for example mannitol, glucose, dextrose, albumins or similar, whereas essentially physiological saline or a 5% glucose solution serves as diluent. Furthermore, it is usual to buffer the solutions with suitable reagents, for example phosphates. In addition, any other additives usually employed for the preparation of pharmaceutical agents can be added, provided the composition comprising the organic vehicle and the compounds according to the invention is not adversely affected.

However, as a result of conversion to duplex active substances, not only can the resistance to enzymatic hydrolysis be increased and the application forms greatly extended, but surprisingly, cytostatic and virostatic effects can also be optimized.

The duplex active substances can be used against malignant diseases of the hematopoietic cells and solid tumors. Owing to the improved cytostatic action, there are far fewer serious side effects. Higher doses of the cytostatically active compounds according to the invention can be used and therapy can be applied in time intervals.

In particular, the compounds according to the invention can be used for the prophylaxis and/or therapy of the following neoplastic diseases: leukemia, lung cancer, intestinal cancer, cancer of the central nervous system, melanomas, ovarian cancer, renal cancer, prostate cancer and breast cancer.

Surprisingly the duplex active substances according to the invention also display virostatic effects, so that they can be used in chemotherapy of virus-induced infections and for overcoming resistance to medicinal products.

In particular the compounds according to the invention can be used for the prophylaxis and/or therapy of the following viral diseases: AIDS (HIV infection), hepatitis A, B and C, herpes and CMV infections.

The invention will now be described in greater detail with the following nonlimiting examples. Unless stated otherwise, the production, formulation and testing of active substances and compositions according to the invention are carried out using current methods of the prior art.

### EXPERIMENTAL SECTION

### Example 1

### Preparation of 5-fluoro-2′-deoxyuridyl-(3′-5′-3′-C-ethynylcytidine

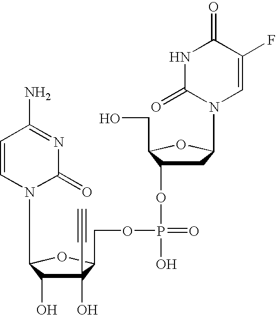

### a) Production of Educts

### Educt 1:

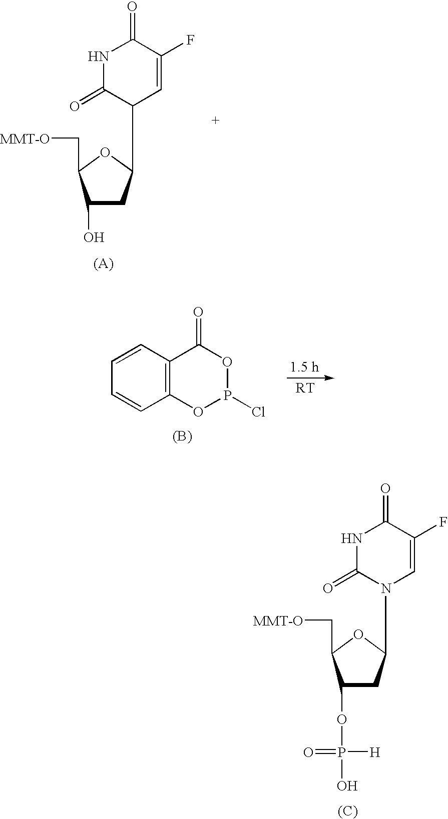

5′-O-(4-Monomethoxytrityl)-5-fluoro-2′-deoxyuridine-3′-hydrogenphosphonate (C) is produced in a two-stage process. First 5′-O-(4-monomethoxytrityl)-5-fluoro-2′-deoxyuridine (A) is prepared as in Ludwig, P. S. et al., European Journal of Medical Chemistry 2005 494-504 (cf. compound (1) there). Then the 3′-hydrogen phosphate group is introduced in the following way. For this, a solution of 20 g (39 mmol) of 5′-0-(4-monomethoxytrityl)-5-fluoro-2′-deoxyuridine (A) in 50 ml of anhydrous pyridine is diluted with 90 ml of anhydrous dioxane and then 50 ml dioxane, in which 11 g (54 mmol) of salicyl chlorophosphite (B) is dissolved, is added. The reaction mixture is stirred at room temperature for 1.5 h. The resultant precipitate is then removed by suction and washed with cold ether. The filtrate and wash liquid are combined, 50 ml saturated sodium carbonate solution is added and concentrated in a rotary evaporator to a foam, which is then dissolved in approx. 300 ml of chloroform/methanol mixture (95:5), fractionated in a silica gel column with a chloroform/methanol gradient, with increasing proportion of methanol. The combined product-containing fractions are concentrated in a rotary evaporator to a foam and yield, after vacuum drying, 20.5 g (35 mmol) of 5′-O-(4-monomethoxytrityl)-5-fluoro-2′-deoxyuridine-3′-hydrogenphosphonate (C).

### Educt 2:

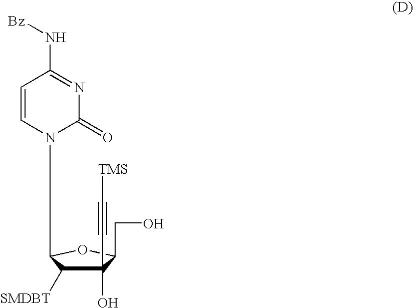

N⁴-Benzoyl-2′-O-(tert.-butyldimethylsilyl)-3′-C-(trimethylsilylethynyl)cytidine (D) is prepared according to the synthesis specification in Ludwig, P. S. et al., Synthesis 2002, 16, 2387-2392 (cf. compound (6) there).

### b) 3′-5′-Coupling of the Educts

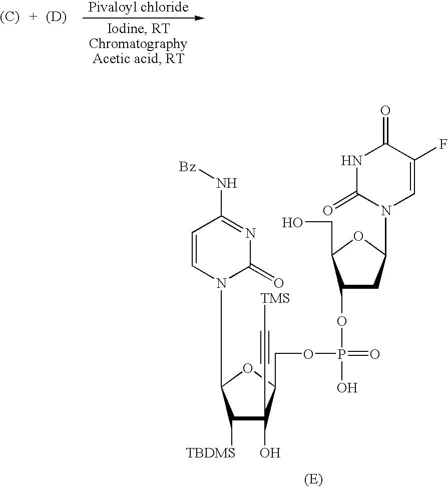

19.2 g (33 mmol) of 5′-O-(4-monomethoxytrityl)-5-fluoro-2′-deoxyuridine-3′-hydrogenphosphonate (C) is dissolved together with 18.4 g (33 mmol) of N⁴-benzoyl-2′-O-(tert.-butyldimethylsilyl)-3′-C-(trimethylsilylethynyl)cytidine (D) in 100 ml of anhydrous pyridine. After the solution has cooled to approx. 10° C., 24 ml (195 mmol) of pivaloyl chloride is added, with exclusion of moisture, the solution is shaken vigorously for 5 min at room temperature, then cooled rapidly to approx. 0° C. and 20 ml of water is added. The reaction mixture is stirred for a few minutes at room temperature, then 160 ml of a solution of 25.4 g iodine in 450 ml tetrahydrofuran is added and then it is stirred for 1 h at room temperature. Excess iodine is reduced by adding solid NaHSO₃, before the reaction mixture is concentrated in a rotary evaporator to a syrup, which is then taken up in 300 ml of chloroform/methanol mixture (9:1) and is extracted with approx. 250 ml of water. The organic phase is concentrated in the rotary evaporator to a syrup, which is diluted with 300 ml of chloroform and is then fractionated in a silica gel column with a chloroform/methanol gradient, with increasing proportion of methanol. In the course of chromatography, the monomethoxytrityl group is already replaced with hydrogen in some of the product. The fractions containing the product with and without monomethoxytrityl residue yield, after concentration in the rotary evaporator, approx. 32 g of foam. For complete replacement of the monomethoxytrityl group with hydrogen, the foam obtained is stirred together with 50 ml methanol in 60 ml of 80% acetic acid for 24 h at room temperature and then concentrated again in the rotary evaporator to a foam. Approximately 120 ml of ether is added to the foam, it is shaken vigorously, converting it to a precipitate, which yields, after centrifugation and drying, approx. 25 g of a solid. The solid is dissolved in approx. 250 ml of chloroform and is fractionated in a silica gel column with a chloroform/methanol gradient, with increasing proportion of methanol. The product-containing fractions are concentrated in the rotary evaporator to a foam, which on adding ether is transformed to a solid (E), which after drying yields 19 g.

### c) Preparation of the End Product

For replacement of the silyl groups with hydrogen, the solid (E) obtained according to stage b) is dissolved in 170 ml of dry tetrahydrofuran, 85 ml of 1M solution of tetrabutylammonium fluoride trihydrate in tetrahydrofuran is added, it is sealed and stirred for 3 days at room temperature and is then concentrated in the rotary evaporator to a syrup, obtaining compound (F)

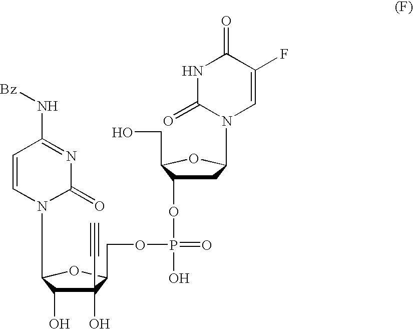

For subsequent replacement of the benzoyl residue with hydrogen, approx. 300 ml of 33% ammonia solution is added to the syrup obtained and, while sealed, it is stirred for 5 days at room temperature. The mixture is then concentrated to approx. 250 ml in the rotary evaporator. The resultant fine precipitate is centrifuged off. The supernatant is lyophilized. The lyophilizate is dissolved in approx. 60 ml of water and is fractionated in a preparative RP-18 column with a water/methanol gradient, with increasing proportion of methanol. The product-containing fractions, which leave the column at a proportion of methanol in the gradient of approx. 15%-40%, are combined, adjusted to pH approx. 5.8 with a cation exchanger (H⁺ form) and separated from the exchanger. The filtrate is neutralized with ammonia, then concentrated and then lyophilized. We obtain 10.6 g of the desired product as tetrabutylammonium salt. The calculated molecular weights for the anionic form 574.40 and the tetrabutylammonium salt form 815.8 are confirmed in the FAB mass spectrum by the molecular peaks 574.0 and 815.8 [M-H]⁻.

### Example 2

### Preparation of 5-fluoro-2′-deoxyuridylyl-(5′-5′)-3′-C-ethynylcytidine

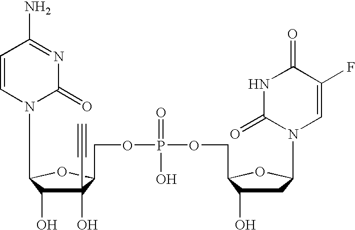

### a) Preparation of the Educts:

### Educt 1:

3′-4-Di-O-benzoyl-5-fluoro-2′-deoxyuridine-5′-hydrogenphosphonate is prepared from 5′-O-(4-monomethoxytrityl)-5-fluoro-2′-deoxyuridine in a two-stage process.

For this, 26 g (50 mmol) of 5′-O-(4-monomethoxytrityl)-5-fluoro-2′-deoxyuridine is dissolved in 150 ml of anhydrous pyridine. Then 56 g (400 mmol) of benzoyl chloride is added to the cooled solution and it is stirred, with exclusion of moisture, at room temperature for 8 h. The reaction is stopped by adding 130 ml of saturated sodium carbonate solution to the cooled solution. The reaction mixture is then concentrated in a rotary evaporator to a syrup, which is diluted with 300 ml of chloroform and is then extracted with 130 ml of saturated sodium carbonate solution. The separated chloroform phase is concentrated to a syrup, which is then diluted with 200 ml of chloroform/petroleum ether mixture (1:1) and is chromatographed in a silica gel column. During this, the column is eluted with a chloroform/petroleum ether gradient with increasing proportion of chloroform. The combined product-containing fractions yield, after concentration under vacuum, approx. 46 g of foam.

For replacement of the 5′-O-(4-monomethoxytrityl) group with hydrogen, the foam is taken up in 100 ml of acetone, in which 16 g of toluene sulfonic acid monohydrate is dissolved. After stirring for 20 min at room temperature, 50 ml of saturated sodium carbonate solution is added to the reaction mixture, and it is concentrated under vacuum to a syrup, which is then diluted with 500 ml of chloroform and 100 ml of water. The separated chloroform phase is fractionated in a silica gel column, elution being carried out first with a chloroform/petroleum ether gradient with increasing proportion of chloroform and then with ether. The combined product-containing fractions are concentrated in a rotary evaporator to a foam, which, after vacuum drying, yields 21 g (46 mmol) of 3′-4-di-O-benzoyl-5-fluoro-2′-deoxyuridine.

For introduction of the 5′-hydrogenphosphonate group, the foam is dissolved in 90 ml of anhydrous pyridine. The solution is diluted with 180 ml of anhydrous dioxane, then a further 75 ml of dioxane is added, in which 13 g (64 mmol) of salicyl chlorophosphite is dissolved, and it is then stirred for 2 h at room temperature. 12 ml of saturated sodium hydrogencarbonate solution is added to the reaction mixture, then it is concentrated under vacuum to a syrup, which is taken up in 500 ml of chloroform and is extracted three times with in each case 200 ml of water/saturated sodium chloride solution/methanol mixture (1:1:2). The chloroform phase is concentrated to a syrup, which is diluted with 150 ml of chloroform and is added, while stirring, to 1.5 l of ether. The resultant precipitate is removed by suction, dried and then extracted with ether for approx. 70 h, leaving 20 g (39 mmol) of 3′-4-di-O-benzoyl-5-fluoro-2′-deoxyuridine-5′-hydrogenphosphonate.

### Educt 2:

N⁴-Benzoyl-2′-O-(tert.-butyldimethylsilyl)-3′-C-(trimethylsilylethynyl)cytidine is prepared according to the synthesis specification in Ludwig, P. S. et al., Synthesis 2002, 16, 2387-2392 (cf. compound (6) there).

### b) 5′-5′-Coupling of the Educts

11.0 g (21.2 mmol) of 3′-4-di-O-benzoyl-5-fluoro-2′-deoxyuridine-5′-hydrogenphosphonate is dissolved, together with 9.5 g (17 mmol) of N⁴-benzoyl-2′-O-(tert.-butyldimethylsilyl)-3′-C-(trimethylsilylethynyl)cytidine, in approx. 100 ml of anhydrous pyridine. By analogy with stage b) in example 1, condensation is started by adding 13 ml (10.6 mmol) of pivaloyl chloride, the reaction is stopped after 5 min by adding 10 ml of water and the condensate is then oxidized with 82 ml of a solution of 25.4 g iodine in 450 ml tetrahydrofuran. After processing the reaction mixture, chromatographic purification in a silica gel column and subsequent ether precipitation, 13 g of a solid is obtained.

### c) Production of the End Product

In the solid obtained according to b), by analogy with stage c) in example 1, first the silyl groups are exchanged for hydrogen, by treating the solid with 45 ml of tetrahydrofuran and 22 ml of 1M solution of tetrabutylammonium fluoride trihydrate in tetrahydrofuran for 3 days at room temperature, and then concentrating to a syrup. Then, for replacing the benzoyl residues with hydrogen, the syrup is stirred, sealed, in 80 ml of 33% ammonia solution for 5 days. The processed reaction mixture is lyophilized and the lyophilizate obtained is fractionated, as described in stage c) of example 1, in a preparative RP-18 column. The product fractions are transformed to 5.8 g of lyophilizate. The calculated molecular weights for the anionic form 574.40 and the tetrabutylammonium salt form 815.8 are confirmed in the FAB mass spectrum by the molecular peaks 574.0 and 815.8 [M-H]⁻.

### Test Example 1

### Determination of the Cytostatic Action In Vitro

The in-vitro cytostatic action of the compounds according to the invention can be demonstrated with the following test setup.

Tumor cell lines, whose 100% growth inhibition (TGI) by the compounds according to the invention is determined at various concentrations, serve as the test system. The toxicity (LC₅₀) of the compound to these cells is also determined. On day 0, a series of microtiter plates is inoculated with the tumor cells and preincubated for 24 h. Then the compound according to the invention is added to the cells in five in each case 10-fold diluted concentration, starting from the highest soluble concentration. After incubation for 48 hours, the cells are fixed in situ, washed and dried. Then sulforhodamine B (SRB), a pink dye that binds to the fixed cells, is added and the cells are washed again. The dye that remains represents the adherent cell mass and is determined spectroscopically. The automatically acquired data are evaluated by computer and lead, for the compound according to the invention 5-fluoro-2′-deoxyuridylyl-(5′-5′)-3′-C-ethynylcytidine and 5-fluoro-2′-deoxyuridylyl-(3′-5′)-3′-C-ethynylcytidine, to the following results, the data for the 3′-5′-coupled dimer being shown in bold above the data for the 5′-5′-coupled isomer.

In-vitro test results of

| | | |
|---|---|---|
| Tumor cell line | TGI in mol/l | LC₅₀ in mol/l |

Lung

| | | |
|---|---|---|
| A549/ATCC | 2.96E−5 | >1.00E−4 |
| | >1.00E−4 | >1.00E−4 |
| EKVX | 6.37E−6 | >1.00E−4 |
| | >1.00E−4 | >1.00E−4 |
| HOP-92 | 2.18E−6 | >1.00E−4 |
| | 2.15E−5 | >1.00E−4 |
| NCI-H522 | 1.15E−6 | >1.00E−4 |
| | >1.00E−4 | >1.00E−4 |

Intestine

| | | |
|---|---|---|
| HCC-2998 | 1.09E−7 | 1.73E−6 |
| | 2.95E−6 | 5.72E−5 |
| HCT-15 | 1.51E−5 | >1.00E−4 |
| | >1.00E−4 | >1.00E−4 |

Central nervous system

| | | |
|---|---|---|
| SF-295 | 1.07E−6 | >1.00E−4 |
| | 2.06E−5 | >1.00E−4 |
| SF-539 | 1.67E−7 | 3.22E−5 |
| | 2.01E−6 | >1.00E−4 |
| SNB-75 | 8.82E−7 | 4.72E−6 |
| | >1.00E−4 | >1.00E−4 |

Melanoma

| | | |
|---|---|---|
| LOX IMVI | 3.48E−7 | 9.15E−5 |
| | 1.00E−4 | 1.00E−4 |
| SK-MEL-2 | 4.24E−7 | 6.95E−5 |
| | 5.78E−6 | 7.64E−5 |

Ovaries

| | | |
|---|---|---|
| IGROV1 | 1.29E−5 | >1.00E−4 |
| | >1.00E−4 | >1.00E−4 |

Kidneys

| | | |
|---|---|---|
| ACHN | 1.52E−6 | >1.00E−4 |
| | >1.00E−4 | >1.00E−4 |
| CAK-1 | 8.29E−8 | >1.00E−4 |
| | >1.00E−4 | >1.00E−4 |

Prostate

| | | |
|---|---|---|
| DU-145 | 4.40E−7 | >1.00E−4 |
| | >1.00E−4 | >1.00E−4 |

Breast

| | | |
|---|---|---|
| MCF7 | 1.37E−7 | >1.00E−4 |
| | >1.00E−4 | >1.00E−4 |
| BT-549 | 3.46E−7 | 6.39E−6 |
| | 1.16E−5 | >1.00E−4 |
| T-47D | 2.67E−5 | >1.00E−4 |
| | >1.00E−4 | >1.00E−4 |

It can be seen that the 3′-5′-coupled duplex active substance (example 1) is much more effective than the 5′-5′-coupled duplex active substance (example 2).

### Test Example 2

### Determination of the In-Vivo Antitumor Activity in the LOX IMVI Melanoma Xenograft Model

The duplex active substance according to the invention, produced according to example 1, was tested for efficacy in the established LOX IMVI xenograft model for solid tumors.

### a) Test Procedure:

A cell suspension of 5×10⁶ tumor cells (LOX IMVI, cell line 01/A/1) was implanted subcutaneously in female athymic nude mice (Animal Production Area, Frederick, Md.). Intraperitoneal administration of the active substance began 3 days after tumor implantation. For this, the active substance was administered dissolved in 10% DMSO/common salt solution plus Tween® 80.

Three treatment groups each comprising eight animals received the active substance at doses of 25.0, 16.75 or 11.2 mg/kg per injection. The control group, comprising sixteen mice, received corresponding volumes of injection solution without active substance. Administration was carried out in each case over a four-day period and comprised a total of 5 treatments. The tests were evaluated by determining the T/C values.

For a model based on solid tumors, a T/C value of 40 must be reached to be regarded as effective.

### b) Result:

Treatment with 25 mg/kg/injection was toxic. The treatment group with 16.75 mg/kg/injection showed a maximum T/C value of 7 on day 11 after tumor implantation. The treatment group with 11.2 mg/kg/injection showed an optimum T/C value of 16 on day 13.

It was therefore found that the tested 3′-5′-coupled duplex active substance (example 1) possesses significant in vivo activity in the present tumor model.

### Concluding Comment on Test Examples 1 and 2:

The test results provide unambiguous evidence of the surprising finding that 3′-5′-coupled duplex active substances according to the invention possess in vitro and in vivo antitumor activity. Moreover, the 3′-5′-coupled duplex active substances are significantly more effective than the corresponding 5′-5′-coupled duplex active substances. This latter finding is all the more surprising because a person skilled in the art would not expect the introduction of a sterically possibly hindering ethynyl group in a prodrug, which has a pair of active substances linked via a natural phosphodiester bridge (i.e. 3′-5′-phosphodiester bridge), to display no inhibitory influence on the cleavage of the phosphodiester bond with release of the active substances.

Reference is expressly made to the disclosure of the publications cited herein.

1. Ethynylated heterodinucleoside phosphate analogs of formula I 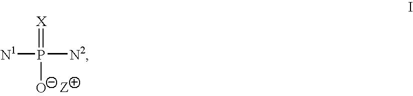
in which
X stands for O or S;
Z stands for H or the corresponding salt of acid addition of this compound;
N¹ and N² are different and in each case stand for a nucleoside group, characterized in that each of the nucleoside groups, which in each case have a glycosidic, or cyclic residue derived therefrom and a basic residue coupled covalently to it, is joined via its glycosidic residue to the central P-atom covalently via a ring-end coupling; and characterized in that at least one of the nucleoside groups has an ethynylated glycosidic residue.

2. The compounds as claimed in claim 1, characterized in that each of the identical or different, optionally ethynylated glycosidic residues of N¹ and N² is derived from a furanose, pentose, hexose or heptose, one or more ring-bound H atoms or hydroxyl groups being optionally eliminated or substituted with H, halogen, hydroxyl, cyano, 2-fluoromethylene, trifluoromethyl or azido; optionally a heteroatom, selected from S, N and O instead of a ring-carbon atom can be contained in the glycosidic residue; and the glycosidic residue can optionally contain one or two nonadjacent C═C double bonds.

3. The compounds as claimed in claim 1, characterized in that each of the identical or different basic residues is the residue of a mono- or binuclear heterocyclic base, which is constructed from one or two four- to seven-membered rings, the basic residue containing at least one basic ring-nitrogen atom and optionally at least one basic amino group and optionally at least one further ring-heteroatom, selected from S and O; and where the basic residue is optionally substituted one or more times with hydroxyl, amino, halogen, alkyl, alkenyl, polyoxyalkenyl, aryl, acyl, alkyloxy, alkenyloxy, polyoxyalkenyloxy, acyloxy, aryloxy, alkylthio, alkenylthio, acylthio or arylthio; the amino, alkyl, alkenyl and acyl residues being optionally substituted with 1 to 3 aryl residues, polyoxyalkylene residues or halogen atoms.

4. The compounds as claimed in claim 3, characterized in that N¹ and N² are P-coupled via identical or different positions of the glycosidic groups.

5. The compounds as claimed in claim 3, characterized in that each of the identical or different glycosidic residues is a furanoside residue or a five-membered residue derived therefrom.

6. The compounds as claimed in claim 5, characterized in that N¹ and N² have a 3′-5′ (ring-end) coupling via the P-atom.

7. The compounds as claimed in claim 5, in which X and Z have the meanings given above, and the groups N¹ and N² are different from one another and stand for a D- or L-configured nucleoside derivative of formula II, III, and IV 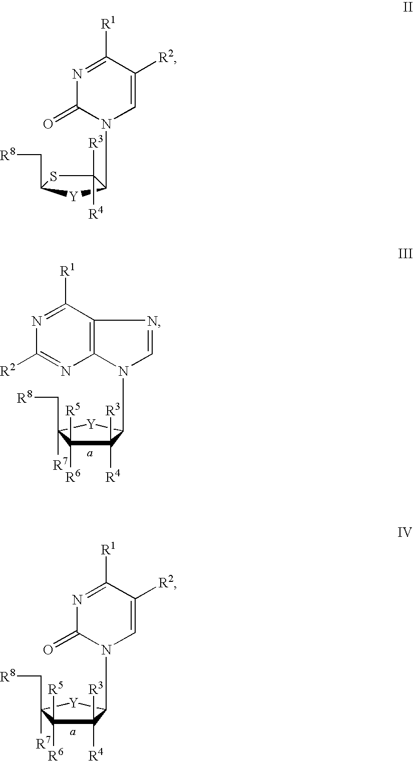
in which
Y stands for O or S;
R¹ represents a hydroxyl, alkoxy, amino, acylated, alkylated or polyoxyethylene-substituted amino group, whose acyl or alkyl residue is linear or branched, has 1 to 24 carbon atoms and up to 2 double bonds and can be substituted with 1-3 aromatic residues or a heterocycle,
R² stands for H, halogen, an amino, hydroxyl or trifluoromethyl group, a bromovinyl, a linear or branched C₁-C₂₄ alkyl residue;
R³ to R⁸ are identical or different, and stand for H, halogen, hydroxyl, ethynyl, cyano, fluoromethylene, trifluoromethyl or azido, with two of the residues R³ to R⁶ being omitted when the C—C bond in position “a” stands for a double bond;
characterized in that N¹ and N² are selected in such a way that always one of the residues R³ to R⁸ of N¹ and N² independently of one another stands for —O— or —S—, via which N¹ and N² are end-ring-coupled to the central P-atom of formula I; and
at least one of the remaining residues R³ to R⁸ in N¹ or N² denotes ethynyl, so that either N¹ or N² has at least one ethynyl residue.

8. The compounds as claimed in claim 7, characterized in that X, Y, Z and “a” have the meanings given above and
a) in groups N¹ and N² independently of one another
R¹ stands for an alkylated or acylated amino group according to the above definition;
R² stands for H, halogen, methyl, ethyl or trifluoromethyl;
R³, R⁴ and R⁷ stand for azido, H, fluoro, fluoromethylene, cyano, trifluoromethyl or hydroxyl;
and simultaneously
b) one of the groups N¹ and N² is ethynylated, and in this group R⁵ stands for ethynyl and in the nonethynylated group R⁵, if present, stands for azido, H, fluoro or hydroxyl;
and simultaneously
c) in the ethynylated group of N¹ and N²
the residue R⁸ stands for —O— or —S— and the residue R⁶ stands for azido, H, fluoro or hydroxyl; and in the nonethynylated group R⁶ stands for —O— or —S— and the residue R⁸ stands for azido, H, fluoro or hydroxyl.

9. The compounds as claimed in claim 7,
in which Z and “a” have the meanings given above X stands for O;
N¹ and N² are different and stand for a nucleoside derivative of formula IV, in which Y stands for O;
R¹ stands for an amino, C₁₂-C₂₂ alkylamino, C₁₂-C₂₂ acylamino group or a hydroxyl group;
R² stands for H, fluoro or trifluoromethyl; and
R³ to R⁸ have the meanings given above.

10. The compounds as claimed in claim 7, characterized in that N¹ stands for an optionally ethynylated nucleoside residue of formula IV, in which the residues R¹ to R⁵, R⁷ and R⁸ have the meanings given above and R⁶ stands for an oxygen atom, with which N¹ is bridged with P, and N² stands for a nucleoside residue of formula IV, which is ethynylated, in which
R¹ stands for amino
R², R³, R⁷ for H;
R⁴, R⁶ for hydroxyl;
R⁵ for ethynyl; and
R⁸ stands for an oxygen atom, with which N² is bridged with P.

11. The compounds as claimed in claim 10, characterized in that N² stands for a nucleoside residue of formula IV, which is ethynylated, and N¹ stands for a nucleoside residue of formula IV, which is not ethynylated, in which
R¹ stands for hexadecyl, palmitoyl, oleoylamino or hydroxyl;
R² stands for H or fluoro;
R³, R⁴ are identical or different and stand for H, hydroxyl, fluoro;
R⁵, R⁷ stands for H;
R⁶ stands for —O— and R⁸ stands for hydroxyl, azido or H.

12. The compounds as claimed in claim 11, characterized in that N² stands for a nucleoside residue of formula IV, which is ethynylated, and N¹ stands for a nucleoside residue of formula IV, which is not ethynylated, in which
R¹ stands for hydroxyl;
R² stands for fluoro;
R³, R⁴, R⁵ and R⁷ stand for H,
R⁶ stands for —O—, and
R⁸ stands for hydroxyl.

13. The compounds as claimed in claim 1, selected from the group consisting of
(a) 5-fluoro-2′-deoxyuridylyl-(3′-5′)-3′-C-ethynylcytidine
(b) (E)-2′-deoxy-(2-fluoromethylene)cytidylyl-(3′-5′)-3′-C-ethynylcytidine
(c) 2′-C-cyano-2-deoxyarabinocytidylyl-(3′-5′)-3′-C-ethynylcytidine
(d) 2-chloro-(2′-deoxy)-fluoroarabinoadenylyl-(3′-5′)-3′-C-ethynylcytidine and
(e) 2′-deoxy-2′,2′-difluorocytidylyl-(3′-5′)-3′-C-ethynylcytidine

14. A pharmaceutical agent, containing at least one compound as claimed in claim 1 in a pharmaceutically compatible vehicle or diluent.

15. The agent as claimed in claim 14, contained in liposomes or nanoparticles.

16. The agent as claimed in claim 14 or 15, additionally containing at least one other pharmacological active substance, which is suitable for the treatment of infectious diseases and/or cancers.

17. A method for the prevention and/or therapy of infectious diseases and/or cancers which comprises administering to a patient in need thereof an effective amount of the agent of claim 14.

18. A method of production of ethynylated heterodinucleoside phosphate analogs as claimed in claim 1, characterized in that two nucleosides of general formulas Va and Vb
L¹-N¹ (Va)
L²-N² (Vb)
in which
N¹ and N² are as defined above and optionally have protecting groups; with at least one of the groups N¹ and N² on the glycosidic residue bearing an ethynyl or protected ethynyl group; and
L¹ and L² on the glycosidic residue of N¹ or N² represent bound, mutually reactive groups, one of the groups L¹ and L² standing for a hydroxy or mercapto group and the other for a hydrogenphosphonate or thiohydrogenphosphonate group, and with one of the groups L¹ and L² bound cyclically and the other bound terminally;
are condensed in the presence of an acid chloride and the condensation product is then oxidized, and optionally present protecting groups are removed.

19. The method as claimed in claim 18, characterized in that in each case two nucleosides of formulas Va and Vb are reacted, characterized in that the nucleosides Va and Vb correspond to a compound of the above general formula II, III or IV, in which
X and “a” have the meanings given above,
L¹ and L² are contained in place of one of the residues R⁶ or R⁸,
the residues R¹ to R⁸ otherwise have the meaning given in claims 7 to 17;
the residues R¹ and R³ to R⁸ additionally also stand for an acylated hydroxyl group, whose acyl residue is linear or branched, has 1-24 carbon atoms and 1 or 2 double bonds and can be substituted with an aromatic residue, or can stand for a tert-butyldimethylsilyloxy protecting group,
R⁸ additionally can also stand for a 4-mono-, or 4,4′-dimethoxytriphenylmethyloxy protecting group;
and residue R⁵ can also stand for trimethylsilylethynyl.

20. The method as claimed in claim 19, characterized in that optionally present 4-mono- or 4,4′-dimethoxytriphenylmethyloxy protecting groups are exchanged for hydroxyl, and acyl and silyl residues are optionally cleaved hydrolytically.
